(19) United States
(12) Patent Application Publication
(10) Pub. No.: US 2008/0014267 A1
(43) Pub. Date: Jan. 17, 2008

(54) Compositions and methods for treatment of coughing, sneezing, rhinorrhea, and/or nasal obstruction

(75) Inventors: John A. Giordano, West Orange, NJ (US); Kevin M. Brown, Harrington Park, NJ (US)

Correspondence Address: Kirkpatrick & Lockhart Preston Gates Ellis LLP;Attn: Ellen Klann, 1601 K St. N.W., WASHINGTON, DC, 20006, US

(73) Assignee: Everett Laboratories, Inc.

(21) Appl. No.: 11/484,750

(22) Filed: Jul. 12, 2006

(51) Int. Cl. A61K 9/22; A61K 31/70; A61K 31/485; A61K 31/137

(52) U.S. Cl. 424/468; 514/282; 514/649; 514/23; 424/725.1

(57) ABSTRACT

The present invention relates to compositions that comprise an expectorant, an extended release antitussive, and an extended release decongestant, in liquid. Specifically, the compositions may comprise guaifenesin, and the tannate salts of phenylephrine and dextromethorphan. The present invention also includes methods for using these compositions for treatment of patients suffering from, for example and without limitation, coughing, sneezing, rhinorrhea, and/or nasal obstruction.

### RELATED APPLICATIONS

This application claims priority under 35 U.S.C. §120 to U.S. patent application Ser. No. 11/330,351, filed Jan. 12, 2006. The entire content of this prior application is fully incorporated herein by reference.

### FIELD OF THE INVENTION

The present invention relates to compositions that comprise, consist of, or consist of essentially an expectorant, an extended release antitussive, and an extended release decongestant in a suspension. Specifically, the compositions comprise guaifenesin, phenylephrine, and dextromethorphan, where phenylephrine and dextromethorphan are extended release forms. Such extended release forms may comprise tannate salts. The present invention also includes methods for using these compositions for treatment of patients suffering from, for example and without limitation, one or more of coughing, sneezing, rhinorrhea, and/or nasal obstruction.

### BACKGROUND OF THE INVENTION

People suffering from coughing, sneezing, rhinorrhea, and/or nasal obstruction commonly take throat lozenges, cough syrups or cough drops for symptomatic relief. While many such medications presently exist, there is room for improvement in the composition of these medications. Many medications contain a combination or variety of antitussives, expectorants and/or decongestants. While a combination or variety may be acceptable to some patients, others may have restrictions due to allergies or other incompatibilities with certain ingredients. Further, many of these medications contain sugar and alcohol while customers would prefer a medication that did not include these substances. Therefore, there is a need for a coughing, sneezing, rhinorrhea, and/or nasal obstruction medication that is free of added sugar and added alcohol that is also restricted to the inclusion of specific antitussives, expectorants, and decongestants.

Guaifenesin is an expectorant which increases the output of phlegm (sputum) and bronchial secretions by reducing adhesiveness and surface tension. The increased flow of less viscid secretions promotes ciliary action and changes a dry, unproductive cough to one that is more productive and less frequent.

Phenylephrine is a sympathomimetic nasal decongestant which acts predominately on alpha adrenergic receptors in the mucosa of the respiratory tract, producing vasoconstriction with minimal action on beta receptors. It functions as an oral nasal decongestant with minimal central nervous system (CNS) stimulation and promotes sinus drainage.

Dextromethorphan acts as an antitussive in suppressing the cough reflex in the medulla. Treatment is intended to relieve cough frequency without abolishing protective cough reflex.

### SUMMARY OF THE INVENTION

The present invention provides compositions and methods of using these compositions for the therapeutic treatment of coughing, sneezing, rhinorrhea, and/or nasal obstruction. Specifically, for example, the present invention relates to novel extended release compositions of antitussives, expectorants, and decongestants that can be used to treat coughing, sneezing, rhinorrhea, and/or nasal obstruction caused by a variety of factors.

In one aspect of the present invention are compositions that comprise guaifenesin, and an extended release form of dextromethorphan, and an extended release form of phenylephrine, in liquid, which is administrable to a patient. In some embodiments, either the extended release dextromethorphan and/or the phenylephrine is a tannate salt.

In some embodiments, the composition is substantially free of other added active ingredients. An other added active ingredient may be one or more of another antitussive, another decongestant, another nasal decongestant, another opioid analgesic or another expectorant.

In some embodiments, the compositions comprise other added active ingredients. An other added active ingredient may be one or more of another antitussive, another decongestant, another nasal decongestant, another opioid analgesic and another expectorant.

In some embodiments of the compositions, the another antitussive may be codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, or menthol.

In some embodiments of the compositions, the another nasal decongestant may be ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, or xylometazoline hydrochloride.

In some embodiments of the compositions, the another opioid analgesic may be codeine, morphine, hydromorphone, hydrocodone, oxymorphone, levorphanol, fentanyl, propoxyphene, diphenoxylate, meperidine, methadone, and oxycodone.

In some embodiments of the compositions, the another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

In another embodiment of the present invention, the compositions may be substantially free of sugar. In another embodiment, the compositions of the present invention may be substantially free of alcohol. In another embodiment of the present invention, the compositions may be substantially free of sugar and substantially free of alcohol. In other embodiments, the composition may be a elixir, syrup and/or suspension.

In another embodiment of the present invention, the compositions may be in a liquid form. In another embodiment of the present invention, the compositions may further comprise a flavorant such as, for example and without limitation, a natural flavorant or an artificial flavorant. Flavorants of the compositions of the present invention may include, for example and without limitation, anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Flavorants included in the compositions of the present invention also may include fruit essence, for example and without limitation, apple essence, pear essence, peach essence, berry essence, wildberry essence, date essence, blueberry essence, kiwi essence, strawberry essence, raspberry essence, cherry essence, plum essence, pineapple essence, and apricot essence. Further, flavorants of the compositions of the present invention also may include, for example and without limitation, natural mixed berry flavor, citric acid, malic acid, vanilla, vanillin, cocoa, chocolate, and menthol.

In a specific embodiment, the compositions of the present invention may also include a non-sugar sweetening agent. For example, the compositions of the present invention may further comprise, without limitation, saccharin sodium, maltodextrin, aspartame, potassium acesulfame, neohesperidin dihydrochalcone, sucralose, monoammonium glycyrrhizinate, and mixtures thereof.

In one embodiment of the present invention, the composition may further comprise citric acid, adetate disodium, glycerin, methylparaben, propylparaben, propylene glycol, saccharin sodium, sodium citrate, sorbitol, water, FD&C red 40, and artificial cherry flavor.

In another embodiment of the present invention, the compositions may comprise about 10 mg/dose to about 30 mg/dose dextromethorphan; about 5 mg/dose to about 15 mg/dose phenylephrine; and/or about 50 mg/dose to about 150 mg/dose guaifenesin.

In another embodiment of the present invention, the compositions may comprise about 15 mg/dose to about 25 mg/dose dextromethorphan; about 7.5 mg/dose to about 12.5 mg/dose phenylephrine; and/or about 75 mg/dose to about 125 mg/dose guaifenesin.

In another embodiment of the present invention, the compositions may comprise about 18 mg/dose to about 22 mg/dose dextromethorphan; about 9 mg/dose to about 11 mg/dose phenylephrine; and/or about 90 mg/dose to about 110 mg/dose guaifenesin.

In another embodiment of the present invention, the compositions may comprise about 19 mg/dose to about 21 mg/dose dextromethorphan; about 9.5 mg/dose to about 11.5 mg/dose phenylephrine; and/or about 95 mg/dose to about 105 mg/dose guaifenesin.

In another embodiment of the present invention, the compositions may comprise about 20 mg/dose dextromethorphan; about 10 mg/dose phenylephrine; and/or about 100 mg/dose guaifenesin.

Another aspect of the present invention includes methods of administering the compositions of the invention to patients. A method comprising administering to a patient a composition comprising compositions that comprise guaifenesin, and an extended release form of dextromethorphan, and an extended release form of phenylephrine in liquid, which is administrable to a patient. In some embodiments, either the dextromethorphan and/or the phenylephrine is a tannate salt.

In some embodiments, the composition of the invention may be administered to the patient orally. In some embodiments, the composition may be administered to the patient at a frequency of once a day, twice a day, three times a day or four times a day. In some embodiments, the composition may be administered to the patient in a dose unit from about 0.1 ml to about 100 ml.

In some embodiments, the patient may be suffering from one or more of coughing, sneezing, rhinorrhea, nasal obstruction, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, a respiratory disease, a cold, acute bronchitis, chronic bronchitis, asthmatic bronchitis, bronchiectasis, pneumonia, lung tuberculosis, silicosis, silicotuberculosis, pulmonary cancer, upper respiratory inflammation, pharyngitis, laryngitis, nasal catarrh, asthma, bronchial asthma, infantile asthma, pulmonary emphysema, pneumoconiosis, pulmonary fibrosis, pulmonary silicosis, pulmonary suppuration, pleuritis, tonsillitis, cough hives, and whooping cough.

In some embodiments of the methods, the composition administered is substantially free of other added active ingredients. An other added active ingredient may be one or more of another antitussive, another decongestant, another nasal decongestant, another opioid analgesic or another expectorant.

In some embodiments of the method, the compositions administered comprise other added active ingredients. An other added active ingredient may be one or more of another antitussive, another decongestant, another nasal decongestant, another opioid analgesic and another expectorant.

In some embodiments of the methods, the another antitussive may be codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, or menthol.

In some embodiments of the methods, the another nasal decongestant may be ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, or xylometazoline hydrochloride.

In some embodiments of the methods, the another opioid analgesic may be codeine, morphine, hydromorphone, hydrocodone, oxymorphone, levorphanol, fentanyl, propoxyphene, diphenoxylate, meperidine, methadone, and oxycodone.

In some embodiments of the methods, the another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

In another embodiment of the present invention, the methods may utilize compositions that may be substantially free of sugar. In another embodiment, the methods may utilize compositions that may be substantially free of alcohol. In another embodiment of the present invention, the methods may utilize compositions that may be substantially free of sugar and substantially free of alcohol. In another embodiment of the present invention, the methods may utilize compositions that are at least one of a elixir, syrup and/or a suspension.

In another embodiment of the present invention, the methods may utilize compositions that may include a flavorant such as, for example and without limitation, a natural flavorant or an artificial flavorant. Flavorants included in the compositions of the present invention may include, for example and without limitation, anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Flavorants included in the compositions of the present invention also may include fruit essence, for example and without limitation, apple essence, pear essence, peach essence, berry essence, wildberry essence, date essence, blueberry essence, kiwi essence, strawberry essence, raspberry essence, cherry essence, plum essence, pineapple essence, and apricot essence. Further, flavorants included in the compositions of the present invention also may include, for example and without limitation, natural mixed berry flavor, citric acid, malic acid, vanilla, vanillin, cocoa, chocolate, and menthol.

In a specific embodiment, the compositions of the present invention may also include a non-sugar sweetening agent. For example, the methods may utilize compositions that may include, without limitation, saccharin sodium, maltodextrin, aspartame, potassium acesulfame, neohesperidin dihydrochalcone, sucralose, monoammonium glycyrrhizinate, and mixtures thereof.

In one embodiment of the present invention, the methods may utilize compositions that may include citric acid, adetate disodium, glycerin, methylparaben, propylparaben, propylene glycol, saccharin sodium, sodium citrate, sorbitol, water, FD&C red 40, and artificial cherry flavor.

In another embodiment of the present invention, the methods may utilize compositions that may comprise about 10 mg/dose to about 30 mg/dose dextromethorphan; about 5 mg/dose to about 15 mg/dose phenylephrine; and/or about 50 mg/dose to about 150 mg/dose guaifenesin.

In another embodiment of the present invention, the methods may utilize compositions that may comprise about 15 mg/dose to about 25 mg/dose dextromethorphan; about 7.5 mg/dose to about 12.5 mg/dose phenylephrine; and/or about 75 mg/dose to about 125 mg/dose guaifenesin.

In another embodiment of the present invention, the methods may utilize compositions that may comprise about 18 mg/dose to about 22 mg/dose dextromethorphan; about 9 mg/dose to about 11 mg/dose phenylephrine; and/or about 90 mg/dose to about 110 mg/dose guaifenesin.

In another embodiment of the present invention, the methods may utilize compositions that may comprise about 19 mg/dose to about 21 mg/dose dextromethorphan; about 9.5 mg/dose to about 10.5 mg/dose phenylephrine; and/or about 95 mg/dose to about 105 mg/dose guaifenesin.

In another embodiment of the present invention, the methods may utilize compositions that may comprise about 20 mg/dose dextromethorphan; about 10 mg/dose phenylephrine; and/or about 100 mg/dose guaifenesin.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

It is understood that the present invention is not limited to the particular methodologies, protocols, fillers, excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a decongestant” is a reference to one or more decongestants and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “patient,” as used herein, comprises any and all organisms and includes the term “subject.” “Patient” may refer to a human or any other animal.

The term “administrable” defines a composition that is able to be given to a patient. Likewise, “administering” refers to the act of giving a composition to a patient or otherwise making such composition available to a patient.

The term “dose” or “dosage,” as used herein, is the amount of medication to be taken at one time.

The term “dose unit,” as used herein, is the amount of the dosage form that is intended to be consumed to deliver the predetermined dosage to an adult human. For example, a dosage unit may be 1 or 2 caplets.

The term “dosage form,” as used herein, is the form in which the dose is to be administered to the patient. The drug is generally administered as part of a formulation that includes nonmedical agents, referred to as pharmaceutic ingredients. The dosage form has a unique physical and pharmaceutical characteristics. Dosage forms may be solid, liquid or gaseous. Solid forms include, but are not limited to tablets, caplets, lozenges, wafers etc. Liquid dosage forms include, but are not limited to syrups, elixirs, injectable solutions, and intravenous solutions. Gaseous forms include vapors, inhalants, and the like.

The term “active ingredient” as used herein is any ingredient that is an antitussive, a decongestant or an expectorant when taken orally by a patient.

The term “substantially free,” as used herein, means free from therapeutically effective amounts of compounds when administered in suggested dosages, but may include trace amounts of compounds in non-therapeutically effective amounts.

Through the inclusion of an antitussive, a decongestant and an expectorant, the compositions and methods of the present invention may alleviate symptoms, such as coughing, sneezing, rhinorrhea, and/or nasal obstruction caused by a variety of factors. The invention relates to compositions comprising the guaifenesin and extended release forms of dextromethorphan and phenylephrine in a suspension. Specifically, the compositions may comprise guaifenesin, and the tannate salts of dextromethorphan and phenylephrine.

Antitussive drugs may act peripherally to inhibit cough by suppressing the responsiveness of one or more vagal sensory receptors that produce cough. Bolser et al., 86(3) J. APPL. PHYSIOL. 1017-1024 (1999); Adcock, RESPIR. MED. 85, Suppl. A 43-46 (1991); Bolser, 9 PULM. PHARMACOL. 357-364 (1997); Kase, 1 TRENDS PHARMACOL. SCI. 237-239 (1980). Antitussive drugs also may act within the central nervous system at the level of the brain stem, where the basic neural circuitry responsible for cough is located. Bolser et al., supra; Korpas & Tomori, COUGH AND OTHER RESPIRATORY REFLEXES, New York, Karger (1979), Shannon, 9 PULM. PHARMACOL. 343-347 (1997); Shannon et al., NEURAL CONTROL OF BREATHING, edited by Miller et al. 215-224 (1996). Specifically, centrally-acting antitussives are thought to inhibit cough by interfering with the central modulation of afferent signals from the periphery, thereby decreasing sensitivity of the cough center located within the medulla to incoming stimuli. Even more specifically, a recent model of the basic cough circuitry suggests that the eupneic respiratory pattern and the cough motor pattern are produced by essentially the same neural components. Although this pattern generator normally controls breathing, its behavior is modified to produce cough by excitatory inputs from medullary second-order interneurons mediating pulmonary rapidly and slowly adapting receptor (RAR and SAR, respectively)—afferent information. Shannon, supra; Shannon et al., supra. Centrally active antitussive drugs may act at any level within this system. For example, these drugs could suppress the responsiveness of components of the central pathway for transmitting vagal sensory information (second-order interneurons) and/or have more complex effects on the motor pattern generator for cough. Bolser & DeGennaro, 662 BRAIN RES. 25-30 (1994); Bolser et al., 113 BR. J. PHARMACOL. 1344-1348 (1994); Chou & Wang, 223 J. PHARMACOL. EXP. THER. 249-253 (1975).

Dextromethorphan (DXM) is a potent antitussive. Dextromethorphan (d-3-methoxy-N-methyl-morphinan) is the dextro isomer of levomethorphan. It has for some time been used as a cough suppressant and may be useful for pain treatment and protection against nerve cell damage. The drug acts centrally, in a manner independent of that of opioids (e.g., codeine), to elevate the threshold for coughing by an, as yet, ill-defined mechanism. DXM, as well as its metabolite, methorphan, have been shown to be antagonists at the N-methyl-d-aspartate (NMDA) receptor which is linked to other amino acid neurotransmitters, glutamate and glycine. In one specific embodiment, dextromethorphan tannate may be used.

Tannate salts of active agents are known to provide therapeutic effect for longer periods of time. It is believed that the inclusion of an active agent in a tannate salt form extends the release profile of the active agent and there is less spiking in pharmacological effect of the active agent. The active agent can therefore be administered to the patient less often and there are fewer side effects, particularly from over dosage effects. See, U.S. Pat. Nos. 5,599,846, and 5,663,415, issued Feb. 4, 1997 and Sep. 2, 1997, respectively, for further information on the preparation and use of phenylephrine tannate. See, U.S. Pat. Nos. 6,670,370 and 6,509,492, issued Dec. 30, 2003 and Jan. 21, 2003, respectively, for further information on the preparation and use of dextromethorphan tannate. In one embodiment, the tannate salts of phenylephrine or dextromethorphan are prepared in situ according to the method provided in U.S. Pat. No. 6,869,618, issued Mar. 22, 2005. In situ conversion to the tannate salts of the active ingredients allows for the preparation of medications with more accurate dosages of the active ingredients than the traditional methods to prepare tannate salts.

In another specific embodiment of the compositions and methods of the present invention, dextromethorphan may be included in amounts ranging from about 19 mg/dose to about 21 mg/dose. In another specific embodiment of the compositions and methods of the present invention, dextromethorphan may be included in amounts ranging from about 18 mg/dose to about 22 mg/dose. In another specific embodiment of the compositions and methods of the present invention, dextromethorphan may be included in amounts ranging from about 15 mg/dose to about 25 mg/dose. In another specific embodiment of the compositions and methods of the present invention, dextromethorphan may be included in amounts ranging from about 10 mg/dose to about 30 mg/dose. In another specific embodiment of the compositions and methods of the present invention, dextromethorphan may be included in an amount of about 20 mg/ dose.

Phenylephrine ((S)-3-hydroxy-α[(methylamino)methyl]benzene-methanol) is a sympathomimetic amine. It acts as an oral nasal decongestant and laryngeal mucous membrane decongestant, with minimal central nervous stimulation, by stimulating alpha-adrenergic receptors to produce pronounced vasoconstriction in the skin, mucous membranes and the mucosa. In a specific embodiment, phenylephrine may be in the form of phenylephrine tannate. In a specific embodiment of the compositions and methods of the present invention, phenylephrine may be included in amounts ranging from about 9.5 mg/dose to about 10.5 mg/dose. In another specific embodiment of the compositions and methods of the present invention, phenylephrine may be included in amounts ranging from about 9 mg/dose to about 11 mg/dose. In another specific embodiment of the compositions and methods of the present invention, phenylephrine may be included in amounts ranging from about 7.5 mg/dose to about 12.5 mg/dose. In a specific embodiment of the compositions and methods of the present invention, phenylephrine may be included in amounts ranging from about 5 mg/dose to about 15 mg/dose. In another specific embodiment of the compositions and methods of the present invention, phenylephrine may be included in an amount of about 10 mg/dose.

Viscous secretion exists in the airway of the human body. This secretion has an important role in imparting suitable temperature and humidity to inhaled air. When its amount is moderate, the secretion in the airway is unconsciously swallowed or expelled with the breath, but usually never is expectorated. Thus, any expectoration suggests that there is something extraordinary in the respiratory system. On the other hand, accumulation of this secretion in the airway is liable to cause an infection via the airway. From this point of view, the removal of the secretion is a matter of great significance in the medical treatment of patients who suffer with a disease in the airway.

In order to facilitate expectoration, medicines referred to as “expectorants” have been used. Most expectorants serve to remove the secretion by diluting it through an increase in secretion by the mucosa of the airway, promotion of separation from the mucosa and enhancement of ciliary beat. Guaifenesin (3-(2-methoxypphenoxy)-1,2-propanediol), also known as glyceryl guaiacolate, is an expectorant. It is readily absorbed from the intestinal tract and is thought to enter airway secretions unmetabolized and to have a direct effect either on the mucus secretion itself or the epithelium. Rubin, 116 CHEST 195-200 (1999). For example, guaifenesin is thought to reduce the thickness of mucus and phlegm secretions by increasing the production of fluids in the respiratory tract thus helping to liquefy and thin airway secretions. The increased flow of less viscid secretions promotes ciliary action and further facilitates the removal of airway secretions. Guaifenesin also may inhibit cough peripherally in the airway, by hydrating airway mucus so that it shields the cough receptors from cough-inducing irritants. Dicpinigaitis & Gayle, 124 CHEST 2178-2181 (2003). These peripheral actions of guaifenesin aid in the removal of accumulated secretions from the trachea, bronchi and lungs, thus changing a dry, non-productive cough to a cough that is more productive and less frequent. Guaifenesin also may act to suppress cough through an effect in the central nervous system. Rubin, supra. While the exact mechanism of this action of guaifenesin is not known, it is believed that guaifenesin acts centrally by depressing or blocking nerve impulse transmission at the internuncial neuron level of the subcortical areas of the brain, brainstem and spinal cord thus relaxing both the laryngeal and pharyngeal muscles.

In a specific embodiment of the compositions and methods of the present invention, guaifenesin may be included in amounts ranging from about 95 mg/dose to about 105 mg/dose. In another specific embodiment of the compositions and methods of the present invention, guaifenesin may be included in amounts ranging from about 90 mg/dose to about 110 mg/dose. In another specific embodiment of the compositions and methods of the present invention, guaifenesin may be included in amounts ranging from about 75 mg/dose to about 125 mg/dose. In another specific embodiment of the compositions and methods of the present invention, guaifenesin may be included in amounts ranging from about 50 mg/dose to about 150 mg/dose. In another specific embodiment of the compositions and methods of the present invention, guaifenesin may be included in an amount of about 100 mg/dose.

In some embodiments of the present invention, the compositions may be substantially free of active ingredients other than guaifenesin, phenylephrine, and dextromethorphan. In some embodiments, the compositions may be substantially free of extended release forms of phenylephrine and dextromethorphan other than the tannate salts. In some embodiments, the compositions may be substantially free of tannate. For example, in one embodiment, the compositions of the present invention may be substantially free of at least one other added antitussive. In another embodiment of the present invention, the compositions may be substantially free of at least one other added decongestant. In another embodiment of the present invention, the compositions may be substantially free of at least one other added nasal decongestant. In another embodiment of the present invention, the compositions may be substantially free of at least one other added opioid analgesic. In another embodiment of the present invention, the compositions may be substantially free of at least one other expectorant. In other embodiments of the present invention, the compositions may be substantially free of one or more other active ingredient, such as, but not limited to, antitussives, decongestants, nasal decongestants, opioid analgesics, and/or expectorants.

In other embodiments of the present invention, the compositions may additionally comprise one or more added active ingredients in addition to guaifenesin, phenylephrine, and dextromethorphan. For example, in one embodiment, the compositions of the present invention may comprise at least one other added antitussive. In another embodiment of the present invention, the compositions may comprise at least one other added decongestant. In another embodiment of the present invention, the compositions may comprise at least one other added nasal decongestant. In another embodiment of the present invention, the compositions may comprise at least one other opioid analgesic. In another embodiment of the present invention, the compositions may comprise at least one other expectorant. In other embodiments of the present invention, the compositions may comprise one or more other active ingredient, such as, but not limited to, antitussives, decongestants, nasal decongestants, opioid analgesics, and/or expectorants.

Antitussives of interest include, but are not limited to, codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan hydrobromide, chlophendianol hydrochloride, carbetapentane citrate, caramiphen edisylate, noscapine, noscapine hydrochloride, and menthol.

Decongestants of interest include, but are not limited to, ephedrine, ephedrine sulfate, ephedrine hydrochloride, pseudoephedrine hydrochloride, phenylephrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

Opioid analgesics of interest include, but are not limited to, such as, codeine, morphine, hydromorphone, hydrocodone, oxymorphone, levorphanol, fentanyl, propoxyphene, diphenoxylate, meperidine, methadone, oxycodone, butorphanol, benzonate and morphine.

Expectorants of interest include, but are not limited to ammonium chloride, ammonium carbonate, acetylcysteine, antimony potassium tartrate, glycerin, potassium iodide, sodium citrate, terpin hydrate, and tolu balsam.

In some embodiments of the present invention, the compositions may substitute one or more of guaifenesin, phenylephrine and dextromethorphan. For example, in one embodiment, the compositions of the present invention may substitute another expectorant for guaifenesin. In another embodiment of the present invention, the compositions may substitute another decongestant for phenylephrine. In another embodiment of the present invention, the compositions may substitute another antitussive for dextromethorphan

Substitute antitussives of interest include, but are not limited to, codeine, codeine phosphate, codeine sulfate, morphine, morphine sulfate, hydrocodone, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan hydrobromide, chlophendianol hydrochloride, carbetapentane citrate, caramiphen edisylate, noscapine, noscapine hydrochloride, and menthol, and functional variants and derivatives thereof.

Substitute decongestants of interest include, but are not limited to, ephedrine, ephedrine sulfate, ephedrine hydrochloride, pseudoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride, and functional variants and derivatives thereof.

Substitute expectorants of interest include, but are not limited to ammonium chloride, ammonium carbonate, acetylcysteine, antimony potassium tartrate, glycerin, potassium iodide, sodium citrate, terpin hydrate, and tolu balsam.

Sugars found in various products are highly undesirable for a number of reasons. For instance, it is known that products, such as medications, containing a high sugar content, more particularly saccharose, fructose and dextrose, may attack the dental enamel as a result of acid being formed in the mouth by certain bacteria if the teeth are not cleaned properly. In addition, many sugar-containing products are unsuitable for diabetics because of their sugar content. Sugar found in manufactured products also may be undesirable due to the increased caloric content caused by the sugar. Because of these factors, there is a progressively increasing world demand for sugar free products, including medications.

Sugar free versions of products may be manufactured using sugar replacements, such as, for example and without limitation, saccharin sodium, maltodextrin, aspartame, potassium acesulfame, neohesperidin dihydrochalcone, sucralose, monoammonium glycyrrhizinate, and mixtures thereof. These “sugar replacements” have the advantage that they do not decompose to form products that attack the dental enamel as a result of the bacterial flora present in the mouth during metabolism, even if the teeth are not cleaned properly. The “sugar replacements” also are suitable for consumption by diabetics and do not add unneeded or unwanted calories to products such as medications. Thus, in a specific embodiment of the compositions and methods of the present invention, the compositions may be free of any added sugar, and instead, may contain a sugar substitute, such as for example and without limitation, one or more of the sugar replacements described above.

Many cough and decongestant medications also contain alcohol. For various legal, dietary, cultural and religious reasons, there is interest in the production of alcohol-free medications. For instance, many alcohol-containing medications cannot be used by children because parents may be concerned about the alcohol content, and children may reject the alcohol bite and astringency characteristic of these products. Indeed, most alcohol-containing medications display the language “Keep Away From Children” on their labels. Similarly large numbers of adults reject alcohol based medications for various personal reasons. For instance and without limitation, those recovering from alcohol abuse avoid medications containing alcohol because of the threat that these substances can trigger a negative response. In addition, many institutionalized individuals are not allowed to use alcohol based medications. Therefore, there is a need for medications that are free of any added alcohol. In a specific embodiment of the compositions and methods of the present invention, the compositions may be free of any added alcohol.

The compositions and methods of the present invention may alleviate symptoms, such as coughing, sneezing, rhinorrhea, and/or nasal obstruction, caused by a variety of conditions. For instance, coughing, sneezing, rhinorrhea, and/or nasal obstruction may be caused by, for example and without limitation, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, or a respiratory disease, such as, for example and without limitation, a cold, acute bronchitis, chronic bronchitis, asthmatic bronchitis, bronchiectasis, pneumonia, lung tuberculosis, silicosis, silicotuberculosis, pulmonary cancer, upper respiratory inflammation (caused by, for example and without limitation, pharyngitis, laryngitis, nasal catarrh), asthma, bronchial asthma, infantile asthma, pulmonary emphysema, pneumoconiosis, pulmonary fibrosis, pulmonary silicosis, pulmonary suppuration, pleuritis, tonsillitis, cough hives, or whooping cough. The compositions and methods of the present invention may provide relief from symptoms caused by all of the above.

Typically cough and decongestant preparations are administered in a liquid form, such as, but limited to, an elixir in a sweetened aromatic solution of alcohol and water. In the present invention, however, syrups and other liquid vehicles, may be used. The preparation may or may not be a suspension. As used herein, the term “syrup” refers to a concentrated, aqueous preparation of a sugar or sugar substitute with or without an added flavoring agent. As used herein, the term “elixir” refers to a clear, sweetened, hydroalcoholic solution intended for oral use, and may or may not have an added flavoring agent. As used herein, a “suspension” is a preparation containing finely divided drug particles distributed somewhat uniformly throughout a vehicle in which the drug exhibits a minimum degree of solubility. Although water itself may make up the entire carrier, typical cough formulations may contain a co-solvent, for example and without limitation, propylene glycol and/or glycerin, to assist solubilization and incorporation of water insoluble ingredients, flavorants and the like into the composition. Any such ingredients may be included as desired or needed within the compositions and methods of the present invention as long as they are consistent with the objectives herein defined. For example, it is contemplated that when desirable, flavoring, preserving, suspending, thickening and/or emulsifying agents may be included in the compositions and methods of the present invention. Formulations for orally administered medications are well known in the art. Descriptions of suitable formulations may be found in Remington, The Science and Practice of Pharmacy (A. Gennaro ed., 20^{th} ed., Lippincott, Williams & Wilkins, 2000).

Flavorants that may be used in accordance with the present invention include those known to those skilled in the art. These flavorants may include, for example and without limitation, natural, artificial and synthetic flavor oils and flavoring aromatic and/or oils, oleoresins and extracts derived from plants, animals, leaves, flowers, fruits, and so forth, and combinations thereof. Non-limiting representative flavor oils include anise oil, cinnamon oil, peppermint oil, spearmint oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Also useful flavorants include fruit essences including apple essence, pear essence, peach essence, berry essence, wildberry essence, date essence, blueberry essence, kiwi essence, strawberry essence, raspberry essence, cherry essence, plum essence, pineapple essence, and apricot essence. Other useful flavorants include aldehydes and esters such as benzaldehyde (cherry, almond), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), mixtures thereof and the like. Honey and artificial honey flavor, as well as natural mixed berry flavor, citric acid, malic acid, vanilla, vanillin, cocoa, chocolate, and menthol may also be used in accordance with the present invention. Flavorants appealing to non-human patients may also be included in the composition of the invention, including but not limited to, yeast extract, meat extract, fish extract, poultry extract, cheese and other dairy flavors, and the like.

Another aspect of the invention are methods of administering to a patient the composition of the invention, comprising guaifenesin, and extended release forms of phenylephrine and dextromethorphan. In some embodiments, the composition comprises guaifenesin, and the tannate salts of phenylephrine and dextromethorphan. In some embodiments, the composition is administered to the patient orally. The compositions of the invention may be administered in varying volumes and at varying frequencies. In specific embodiments, the dose unit is from about 1 to about 10 ml, or from about 0.1 to 100 ml. Specific dose units include, but are not limited to, 1.25 ml, 2.5 ml, 5.0 ml, and 10 ml. The frequency of the dose may vary from every other day to several times a day. In specific embodiments, the frequency of administration may be once a day, twice a day, three times a day or four times a day. In other specific embodiments, the frequency of the dose may be once a day or twice a day.

The dose may depend in part with the characteristics of the patient. In some embodiments, the patient is a human over about 12 years of age and the composition may be administered in an about one dose at least once a day, at least twice a day, once a day or twice a day. In other embodiments, the patient is human from about 6 to about 12 years of age, and the composition of the invention is administered in an about ½ dose once a day or twice a day. In another embodiment, the patient is a human from about 2 to about 6 years of age, and the composition of the invention is administered in an about ¼ dose once a day or twice a day.

The total dosage per day of the active compounds may be a factor in determining the criteria for administering the composition of the invention. For example, compositions with a higher concentration of active compounds may be taken in smaller dosages and/or less frequently, and compositions with lower concentrations of the active compounds may be taken in larger volume dosages and/or more frequently.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation is prepared in liquid swallowable form containing the following active ingredients per 5 ml:

| | |
|---|---|
| Guaifenesin | 100 mg |
| Phenylephrine, as tannate salt | 10 mg |
| | (20 mg tannate salt) |
| Dextromethorphan, as tannate salt | 20 mg |
| | (40 mg tannate salt) |

Other inactive ingredients include: aspartame, acesulfame, glycerin, neusilin, xanthan gum, sodium citrate dehydrate, citric acid; methylparaben, sodium benzoate, bitter mask, FD&C blue #1, FD&C red #40, artificial grape flavor, purified water, 6N hydrochloric acid, 6N sodium hydroxide.

### Example 2

A study is undertaken to evaluate the effectiveness of the compositions of the present invention in the treatment of patients. The objective of the study is to determine whether oral intake of the compositions of the present invention results in an improvement of the symptoms of coughing, sneezing, rhinorrhea, and/or nasal obstruction.

A double-blind, placebo controlled study is conducted over a three-day period. A total of 120 subjects, all presenting for treatment of symptoms of coughing, sneezing, rhinorrhea, and/or nasal obstruction, are chosen for the study. The patients range in age from 8 to 72 years old.

An initial assessment of the symptoms of each patient is conducted when the patients initially present for treatment. The treating physician rates the severity of the symptoms on a 4-point scale (0: absent; 1: mild; 2: moderate; 3: severe). For inclusion in the study, a patient must be rated with a score of two or above for cough and a total score of at least 5 for the sum of the four selected symptoms.

The 120 subjects chosen for the study are separated into two separate groups of 60. The characteristics of the symptoms between the two groups are comparable. The first group is administered a 5 ml dose of the composition of the present invention every twelve hours for three days. The second group is administered a placebo medication every twelve hours for three days that is similar in all respects to the administered composition except for the exclusion of the active ingredients, dextromethorphan tannate, phenylephrine tannate and guaifenesin. No other medications are taken by the patients during the assessment period.

Patients self-evaluate their symptoms of coughing, sneezing, rhinorrhea, and nasal obstruction using the same 4-point scale (0: absent; 1: mild; 2: moderate; 3: severe) thirty minutes after each dose administration. Patients also note the presence and severity of adverse effects of taking the medication on the 4-point scale. In addition to the initial assessment on day 1, patients are evaluated at the end of day two and day three by the treating physician.

The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at each dose administration, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

This study will demonstrate the efficacy of the composition of the present invention in treating the symptoms of coughing, sneezing, rhinorrhea and nasal obstruction. Regarding potential adverse effects of taking the medication, if there are no significant differences between the two therapeutic groups, this study will demonstrate that the administration of the composition of the present invention is effective at treating symptoms of coughing, sneezing, rhinorrhea, and nasal obstruction, in addition to being well-tolerated by the patients.

While specific embodiments of the present invention have been described, other and further modifications and changes may be made without departing from the spirit of the invention. All further and other modifications and changes are included that come within the scope of the invention as set forth in the claims. The disclosures of all publications cited above are expressly incorporated by reference in their entireties to the same extent as if each were incorporated by reference individually.

What is claimed is:

1. A composition comprising guaifenesin, an extended release form of dextromethorphan, and an extended release form of phenylephrine in liquid, wherein said composition is administrable to a patient.

2. The composition of claim 1, wherein at least one selected from the group consisting of said dextromethorphan and said phenylephrine is a tannate salt.

3. The composition of claim 2, wherein said phenylephrine and said dextromethorphan are tannate salts.

4. The composition of claim 1, wherein said composition is substantially free of other added active ingredients.

5. The composition of claim 4, wherein said other added active ingredient is one or more selected from the group consisting of another antitussive, another decongestant, another nasal decongestant, another opioid analgesic and another expectorant.

6. The composition of claim 1, wherein said composition comprises other added active ingredients.

7. The composition of claim 6, wherein said other added active ingredient is one or more selected from the group consisting of another antitussive, another decongestant, another nasal decongestant, another opioid analgesic and another expectorant.

8. The composition of claims 5 or 7, wherein said another antitussive comprises one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

9. The composition of claim 5 or 7, wherein said another nasal decongestant comprises one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

10. The composition of claim 5 or 7, wherein said another opioid analgesic comprises one or more of the group consisting of codeine, morphine, hydromorphone, hydrocodone, oxymorphone, levorphanol, fentanyl, propoxyphene, diphenoxylate, meperidine, methadone, and oxycodone.

11. The composition of claim 5 or 7, wherein said another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

12. The composition of claim 1, wherein said composition is substantially free of sugar.

13. The composition of claim 1, wherein said composition is substantially free of alcohol.

14. The composition of claim 1, wherein said composition is substantially free of sugar and substantially free of alcohol.

15. The composition of claim 1, wherein said composition is at least one selected from the group consisting of elixir, syrup and suspension.

16. The composition of claim 1, wherein said composition further comprises a flavorant.

17. The composition of claim 16, wherein said flavorant comprises a natural flavorant.

18. The composition of claim 16, wherein said flavorant comprises an artificial flavorant.

19. The composition of claim 17, wherein said flavorant is selected from one or more of the group consisting of anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil.

20. The composition of claim 17, wherein said flavorant is selected from one or more of the group consisting of apple essence, pear essence, peach essence, berry essence, wildberry essence, date essence, blueberry essence, kiwi essence, strawberry essence, raspberry essence, cherry essence, plum essence, pineapple essence, and apricot essence.

21. The composition of claim 17, wherein said flavorant is selected from one or more of the group consisting of natural mixed berry flavor, citric acid, malic acid, vanilla, vanillin, cocoa, chocolate, and menthol.

22. The composition of claim 1, wherein said composition further comprises a non-sugar sweetening agent.

23. The composition of claim 22, wherein said non-sugar sweetening agent is selected from one or more of the group consisting of saccharin sodium, maltodextrin, aspartame, potassium acesulfame, neohesperidin dihydrochalcone, sucralose, and monoammonium glycyrrhizinate.

24. The composition of claim 1, wherein said composition further comprises citric acid, adetate disodium, glycerin, methylparaben, propylparaben, propylene glycol, saccharin sodium, sodium citrate, sorbitol, water, FD&C red 40, and artificial cherry flavor.

25. The composition of claim 1, wherein said composition comprises about 10 mg/dose to about 30 mg/dose dextromethorphan.

26. The composition of claim 1, wherein said composition comprises about 5 mg/dose to about 15 mg/ dose phenylephrine.

27. The composition of claim 1, wherein said composition comprises about 50 mg/dose to about 150 mg/dose guaifenesin.

28. The composition of claim 1, wherein said composition comprises about 15 mg/dose to about 25 mg/dose dextromethorphan.

29. The composition of claim 1, wherein said composition comprises about 7.5 mg/dose to about 12.5 mg/dose phenylephrine.

30. The composition of claim 1, wherein said composition comprises about 75 mg/dose to about 125 mg/dose guaifenesin.

31. The composition of claim 1, wherein said composition comprises about 18 mg/dose to about 22 mg/dose dextromethorphan.

32. The composition of claim 1, wherein said composition comprises about 9 mg/dose to about 11 mg/dose phenylephrine.

33. The composition of claim 1, wherein said composition comprises about 90 mg/dose to about 110 mg/dose guaifenesin.

34. The composition of claim 1, wherein said composition comprises about 19 mg/dose to about 21 mg/dose dextromethorphan.

35. The composition of claim 1, wherein said composition comprises about 9.5 mg/dose to about 10.5 mg/dose phenylephrine.

36. The composition of claim 1, wherein said composition comprises about 95 mg/dose to about 10.5 mg/dose guaifenesin.

37. The composition of claim 1, wherein said composition comprises about 19 mg/dose to about 21 mg/dose dextromethorphan; about 9.5 mg/dose to about 10.5 mg/dose phenylephrine; and about 95 mg/dose to about 10.5 mg/dose guaifenesin.

38. The composition of claim 1, wherein said composition comprises about 20 mg/dose dextromethorphan.

39. The composition of claim 1, wherein said composition comprises about 10 mg/dose phenylephrine.

40. The composition of claim 1, wherein said composition comprises about 100 mg/dose guaifenesin.

41. The composition of claim 1, wherein said composition comprises about 20 mg/dose dextromethorphan; about 10 mg/dose phenylephrine; and about 100 mg/dose guaifenesin.

42. A method comprising administering to a patient the composition of claim 1.

43. The method of claim 42, wherein the composition is administered to the patient orally.

44. The method of claim 42 wherein the composition is administered to the patient at a frequency selected from the group consisting of once a day, twice a day, three times a day and four times a day.

45. The method of claim 42, wherein the composition is administered to the patient in a dose unit from about 0.1 ml to about 100 ml.

46. The method of claim 42, wherein the patient is suffering from at least one condition selected from the group consisting of coughing, sneezing, rhinorrhea, nasal obstruction, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, a respiratory disease, a cold, acute bronchitis, chronic bronchitis, asthmatic bronchitis, bronchiectasis, pneumonia, lung tuberculosis, silicosis, silicotuberculosis, pulmonary cancer, upper respiratory inflammation, pharyngitis, laryngitis, nasal catarrh, asthma, bronchial asthma, infantile asthma, pulmonary emphysema, pneumoconiosis, pulmonary fibrosis, pulmonary silicosis, pulmonary suppuration, pleuritis, tonsillitis, cough hives, and whooping cough.

47. The method of claim 42, wherein at least one selected from the group consisting of said dextromethorphan and said phenylephrine is a tannate salt.

48. The method of claim 47, wherein both said phenylephrine and said dextromethorphan are tannate salts.

49. The method of claim 42, wherein said composition is substantially free of other added active ingredients.

50. The method of claim 49, wherein said other added active ingredient is one or more selected from the group consisting of another antitussive, another decongestant, another nasal decongestant, another opioid analgesic and another expectorant.

51. The method of claim 42, wherein said composition comprises other added active ingredients.

52. The method of claim 51, wherein said other added active ingredient is one or more selected from the group consisting of another antitussive, another decongestant, another nasal decongestant, another opioid analgesic and another expectorant.

53. The method of claims 50 or 52, wherein said another antitussive comprises one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

54. The method of claim 50 or 52, wherein said another nasal decongestant comprises one or more of the group consisting of ephedrine, ephedrine sulfate, ephedrine hydrochloride, psuedoephedrine hydrochloride; epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

55. The method of claim 50 or 52, wherein said another opioid analgesic comprises one or more of the group consisting of codeine, morphine, hydromorphone, hydrocodone, oxymorphone, levorphanol, fentanyl, propoxyphene, diphenoxylate, meperidine, methadone, and oxycodone.

56. The method of claim 50 or 52, wherein said another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

57. The method of claim 42, wherein said composition is substantially free of sugar.

58. The method of claim 42, wherein said composition is substantially free of alcohol.

59. The method of claim 42, wherein said composition is substantially free of sugar and substantially free of alcohol.

60. The method of claim 42, wherein said composition is at least one selected from the group consisting of elixir, syrup and suspension.

61. The method of claim 42, wherein said composition further comprises a flavorant.

62. The method of claim 61, wherein said flavorant comprises a natural flavorant.

63. The method of claim 61, wherein said flavorant comprises an artificial flavorant.

64. The method of claim 62, wherein said flavorant is selected from one or more of the group consisting of anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil.

65. The method of claim 62, wherein said flavorant is selected from one or more of the group consisting of apple essence, pear essence, peach essence, berry essence, wildberry essence, date essence, blueberry essence, kiwi essence, strawberry essence, raspberry essence, cherry essence, plum essence, pineapple essence, and apricot essence.

66. The method of claim 62, wherein said flavorant is selected from one or more of the group consisting of natural mixed berry flavor, citric acid, malic acid, vanilla, vanillin, cocoa, chocolate, and menthol.

67. The method of claim 42, wherein said composition further comprises a non-sugar sweetening agent.

68. The method of claim 67, wherein said non-sugar sweetening agent is selected from one or more of the group consisting of saccharin sodium, maltodextrin, aspartame, potassium acesulfame, neohesperidin dihydrochalcone, sucralose, and monoammonium glycyrrhizinate.

69. The method of claim 42, wherein said composition further comprises citric acid, adetate disodium, glycerin, methylparaben, propylparaben, propylene glycol, saccharin sodium, sodium citrate, sorbitol, water, FD&C red 40, and artificial cherry flavor.

70. The method of claim 42, wherein said composition comprises about 10 mg/dose to about 30 mg/dose dextromethorphan.

71. The method of claim 42, wherein said composition comprises about 5 mg/dose to about 15 mg/dose phenylephrine.

72. The method of claim 42, wherein said composition comprises about 50 mg/dose to about 150 mg/dose guaifenesin.

73. The method of claim 42, wherein said composition comprises about 15 mg/dose to about 25 mg/dose dextromethorphan.

74. The method of claim 42, wherein said composition comprises about 7.5 mg/dose to about 12.5 mg/dose phenylephrine.

75. The method of claim 42, wherein said composition comprises about 75 mg/dose to about 125 mg/dose guaifenesin.

76. The method of claim 42, wherein said composition comprises about 18 mg/dose to about 22 mg/dose dextromethorphan.

77. The method of claim 42, wherein said composition comprises about 9 mg/dose to about 11 mg/dose phenylephrine.

78. The method of claim 42, wherein said composition comprises about 90 mg/dose to about 110 mg/dose guaifenesin.

79. The method of claim 42, wherein said composition comprises about 19 mg/dose to about 21 mg/dose dextromethorphan.

80. The method of claim 42, wherein said composition comprises about 9.5 mg/dose to about 10.5 mg/dose phenylephrine.

81. The method of claim 42, wherein said composition comprises about 95 mg/dose to about 10.5 mg/dose guaifenesin.

82. The method of claim 42, wherein said composition comprises about 19 mg/dose to about 21 mg/dose dextromethorphan; about 9.5 mg/dose to about 10.5 mg/dose phenylephrine; and about 95 mg/dose to about 10.5 mg/dose guaifenesin.

83. The method of claim 42, wherein said composition comprises about 20 mg/dose dextromethorphan.

84. The method of claim 42, wherein said composition comprises about 10 mg/dose phenylephrine.

85. The method of claim 42, wherein said composition comprises about 100 mg/dose guaifenesin.

86. The method of claim 42, wherein said composition comprises about 20 mg/dose dextromethorphan; about 10 mg/dose phenylephrine; and about 100 mg/dose guaifenesin.
